(19) United States
(12) Patent Application Publication
(10) Pub. No.: US 2025/0225334 A1
(43) Pub. Date: Jul. 10, 2025

(54) PATIENT DATA COLLECTION

(71) Applicant: Electronic Referral Manager Inc., Mt. Pleasant, SC (US)

(72) Inventors: Richard Hammer, Tucson, AZ (US); Vidal Graupera, Burlingame, CA (US); Christopher Betsill, Santa Barbara, CA (US); David Bongiovanni, Charleston, SC (US)

(73) Assignee: Electronic Referral Manager Inc., Mt. Pleasant, SC (US)

(21) Appl. No.: 18/404,985

(22) Filed: Jan. 5, 2024

(51) Int. Cl. G06F 40/40; G06F 40/174; G16H 10/60; G16H 40/20

(52) U.S. Cl. CPC G06F 40/40; G06F 40/174; G16H 10/60; G16H 40/20

(57) ABSTRACT

Embodiments include a method for receiving data, the method comprises, responsive to receiving an indication that conversational artificial intelligence, (AI) has been selected, identifying data types for a data field: engaging in a dialog with a user using a large language model (LLM) to collect data for the data field wherein the dialog includes a question presented to a user and an answer provided by the user, receiving data from a user, tracking the user input on a stack, providing a question to a user, receiving an answer from a user, determining if the answer is complete and meets the criteria for the data field, and sending a second question to the user responsive to receiving a complete answer from the user.

### BACKGROUND

The present invention relates to managing healthcare data, and more specifically, to providing patients, healthcare providers and their staff members with an efficient and useful method for completing forms associated with an appointment.

In the healthcare industry, patients often schedule initial appointments, subsequent appointments, follow-up appointments, and referral appointments among others.

For an appointment, patients are often asked to provide data to healthcare providers by using a paper or a digital form. The digital form is often merely a reproduction of a paper form with similar questions and fields to enter patient data.

It is desirable for a system to receive patient data and provide consent forms for signature that effectively provides a positive user experience and operates in an efficient manner.

### SUMMARY

Embodiments of the present invention comprise a method for receiving data, the method comprises, responsive to receiving an indication that conversational artificial intelligence, (AI) has been selected, identifying data types for a data field: engaging in a dialog with a user using a large language model (LLM) to collect data for the data field wherein the dialog includes a question presented to a user and an answer provided by the user, receiving data from a user, tracking the user input on a stack, providing a question to a user, receiving an answer from a user, determining if the answer is complete and meets the criteria for the data field, and sending a second question to the user responsive to receiving a complete answer from the user.

Embodiments of the present invention comprise a system for receiving data, the system comprises a memory, and a processor operative to responsive to receiving an indication that conversational artificial intelligence (AI) has been selected, identify data types for a data field engage in a dialog with a user using a large language model (LLM) to collect data for the data field wherein the dialog includes a question presented to a user and an answer provided by the user, receive data from a user, track the user input on a stack in the memory, provide a question to a user, receive an answer from a user, determine if the answer is complete and meets the criteria for the data field, and sending follow up questions to the user responsive to receiving a complete answer from the user.

Embodiments of the present invention comprise a non-transitory computer-readable medium with instructions for a method for receiving data, the method comprising responsive to receiving an indication that conversational artificial intelligence (AI) has been selected, identifying data types for a data field engaging in a dialog with a user using a large language model (LLM) to collect data for the data field wherein the dialog includes a question presented to a user and an answer provided by the user, receiving data from a user, tracking the user input on a stack, providing a question to a user, receiving an answer from a user, determining if the answer is complete and meets the criteria for the data field, and sending a second question to the user responsive to receiving a complete answer from the user.

### BRIEF DESCRIPTION OF THE DRAWINGS

FIG. 1 illustrates a block diagram of an example computer system.

FIG. 2 illustrates a block diagram of a method for generating and updating a patient record.

FIG. 3 illustrates a block diagram of a method for generating form instances.

FIGS. 4A-4B illustrate a block diagram of an example method for receiving user data.

FIG. 5 illustrates a block diagram of the method described in block 414 of FIG. Ga,

FIG. 6 illustrates a block diagram of the method of processing payments described in block 422 of FIG. 4.

### DETAILED DESCRIPTION

Collecting data from patients can be a challenge because many data collection systems collect data from patients (users) by using an inefficient process. Previous systems presented questions and entry fields that are in practice similar to using a paper form. Often a patient is presented with questions that are not applicable to the patient, which wastes the time of the patient. These forms fail to offer sufficient solutions to intake data when a user fails to answer a question. Sometimes a user fails to answer a question in a way that provides a healthcare provider with information that will help the healthcare provider provide adequate care to the patient. This system allows patients to enter information using a natural conversational or chat interface instead of filling out forms.

The system described herein provides a system that manages and sets appointments for healthcare providers, and provides methods for managing healthcare data entered into forms and consents by, for example a referring healthcare organization or a patient. A trigger event may include, for example, an appointment with a healthcare provider. The system in embodiments uses artificial intelligence (AI) to improve the user experience and increase the efficiency of data collection and setting appointments.

Although this disclosure includes a detailed description of a computing environment, the teachings herein are not limited to the described computing environment. In embodiments, any implementation of a computer environment may be used whether now understood or later developed.

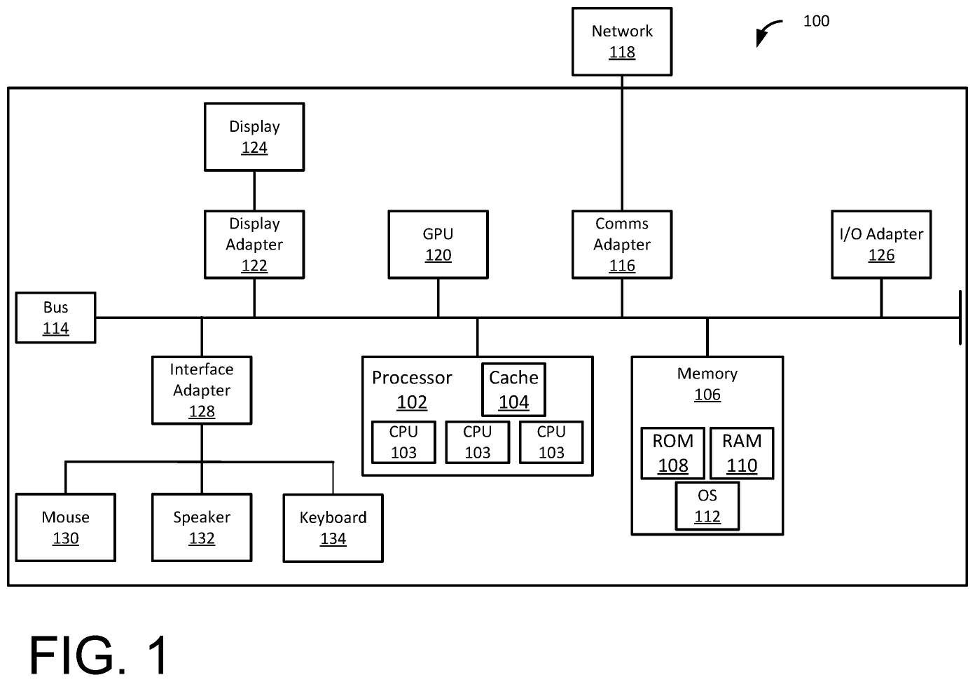

FIG. 1 illustrates a block diagram of an example computer system 100. In embodiments, a microprocessor is arranged in a personal computer, workstation, minicomputer, or mainframe computer. Embodiments of system 100 may include a mobile device or part of a mobile device.

The system (computer) 100 includes processor 102 communicatively connected to an input/output (I/O) adapter 126, memory 106, a communications adapter 116, an interface adapter 128, a display adapter 122, and a graphics processing unit (GPU) 120 via a system bus 114.

The processor 102 includes any number of central processing units (CPU) 103 and a cache memory 104. The CPU 102 is operable to perform any number of processing tasks by executing program instructions. In embodiments the processor 102 may include any number of suitable auxiliary processors or microprocessors. The processor 102 may include a cache memory 104 that is often operative to increase the speed of processing tasks performed by the processor 102.

The I/O adapter 126 connects a variety of input and output devices to the computer 100. The memory 106 may include any type of suitable memory such as, for example, magnetic, solid state, disk-based storage, drum-based storage, random access memory (RAM) 110, and read-only memory (ROM) 108. The operating system of the system 100 may be stored in the memory 106. The communications adapter 116 is operative to facilitate communications between the system 100 and a network 118. The network 118 may include, for example, a local area network, a wide area network or the Internet. The system 100 may connect to the network 118 via, for example, a wired or wireless connection. The display adapter 122 is operative to communicably connect a display 124 to the system 100. The display may include any type of suitable display such as, for example, a desktop display, a touch screen, or a mobile display. The GPU 120 includes a processor that is operative to generate graphical data that may be sent to the display 124 for presentation to a user. The interface adapter 128 provides an interface between peripheral devices and the system bus 114. The peripheral devices may include, for example, a mouse 130, a speaker 132, and a keyboard 134, sensors, and actuators, among others.

The memory 106 may store computer-readable and computer-executable instructions. The instructions implement logical functions. The operating system 112 may control the operations of the system 100 and the functions of other software programs or operations.

Memory can also include program instructions for engine 1, configured to improve the wellness of a user.

According to some embodiments, computer 100 can include a mobile communications adapter 123. Mobile communications adapter 123 can include GPS, cellular, mobile, and/or other communications protocols for wireless communication.

In some embodiments, computer 100 can further include communications adapter 116 for coupling to a network 118.

The present invention may include a computer program, a method, system, and/or a computer program product in any level of integration. A computer program product can comprise of any type of computer-readable storage medium that includes computer-readable program instructions that causes a processor to implement some or all aspects of the invention.

The computer-readable storage medium may include any suitable device that may store instructions for use by a computer, processor or other similar device. The computer-readable storage medium may include, for example, a random access memory, a read-only memory, a flash memory, a magnetic or light readable disk. A computer-readable storage medium as used herein does not include transitory signals such as, for example, electromagnetic waves.

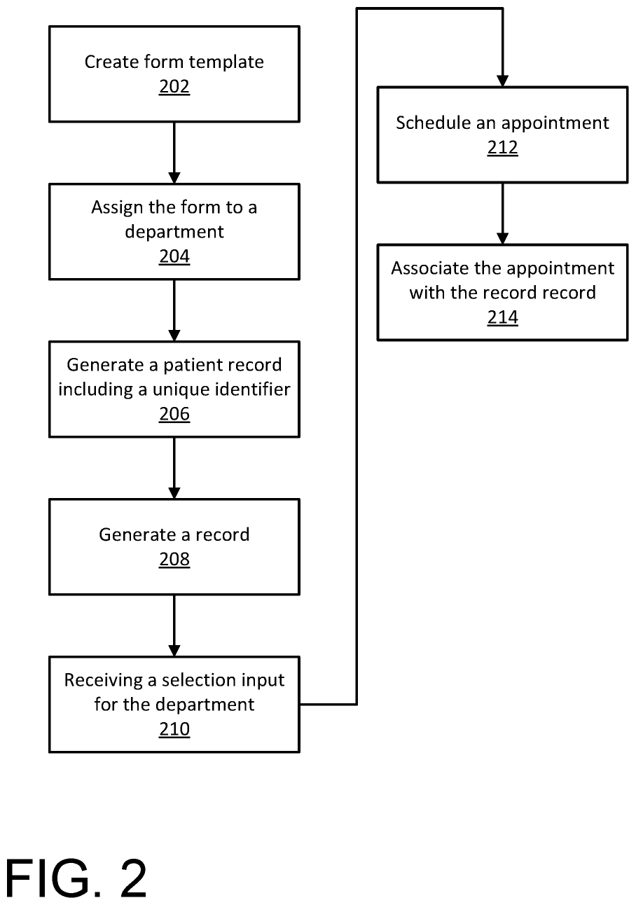

FIGS. 2-4B provide exemplary methods for collecting patient data for healthcare providers.

FIG. 2 illustrates a block diagram of a method for generating a patient record based in part on data collected from a user using a form. In this regard, in block 202 a form template is created. A form template is a customizable user-defined data structure used to define the content of individual form instances. A form template includes, for example, questions that will be presented to the user, a template for dynamic notifications that are sent to the user, and the assignment of identifiers to determine when a form template should be used to create a form instance. Form questions include, for example, a question name, question format, data structures to support the selected question type and flags to determine if a question is applicable. Questions store a relative order of questions in the form. Questions may be nested to create structures where the end-user will be presented with paths of questions with respect to the user responses. Questions may include text that provides additional context with respect to the questions for the user. Questions may also include a value of the response of the user after the response is received by the system 100. The template for dynamic notifications defines the content of the notification or message sent to the user. The template may include dynamic fields including, for example, the user name, appointment time, and appointment location. The notification template includes a generated link that leads to an interface for the user to interact with the system 100. In block 204, the form may be assigned or associated with a healthcare organization or a group in a healthcare organization. In block 206 a unique identifier is associated with the patient record. The unique identifier may include, for example, a user telephone number that may be used to receive text messages.

In block 208 a record that includes data is generated in block 208. A selection input for the assigned department is received in block 210. The selection input or identifiers assigned to the form are used to determine if and when a specific form template should be used. For example, if the identifiers may be used to identify a field that may be linked to an appointment. For example, when an appointment is received for a department, the system 100 determines if there are any form templates assigned to the department. If yes, an instance of the form is created that contains the same questions defined in the form template, a notification is sent to the user with a link to the interface. In block 212 an appointment is scheduled. An appointment may be scheduled by the user or the healthcare provider. In block 214, the appointment is associated with the referral record.

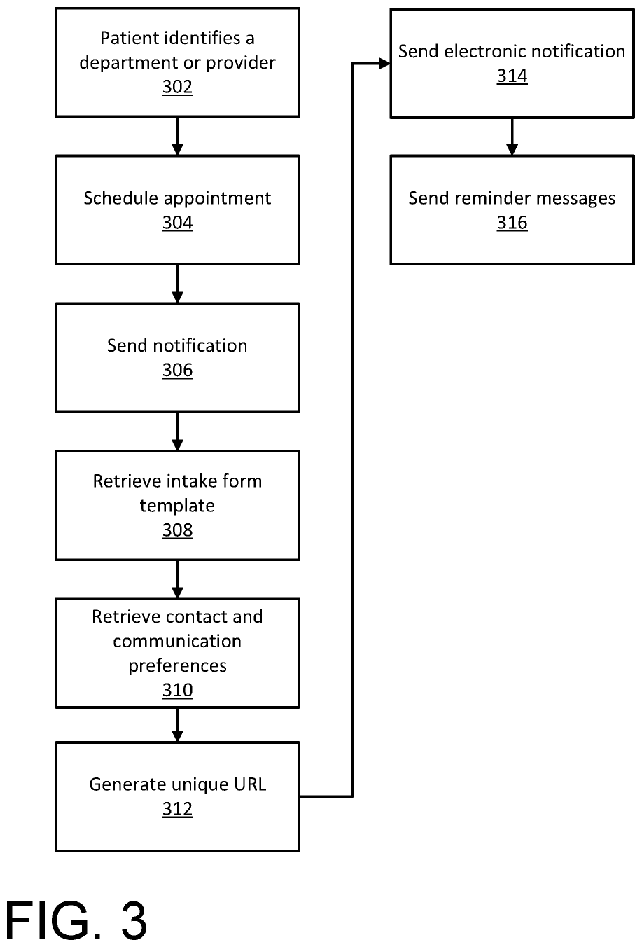

FIG. 3 illustrates a block diagram of a method for generating form templates and sending messages to a patient to prompt the patient to input data. In this regard, in block 302 a patient has selected a healthcare provider or an organization of healthcare providers. In block 304, the system 100 (of FIG. 1) schedules an appointment or referral appointment. In block 306 a notification such as, for example an email or SMS is sent. The notification content is customizable by the system administrators or users. The notification may include a link or URL that may initiate the methods described herein. An intake form template is retrieved from memory in block 308. The intake form template may be tailored in embodiments for a particular healthcare provider or a particular patient. In block 310 contact and communication preferences are retrieved by the system 100. The communication preferences may include, for example, how a patient prefers to be contacted. In block 312 a unique URL is generated that may be used by the patient to access and complete.

In block 314 an electronic notification is sent that includes the URL, and for example, a message with instructions for the recipient. Reminder messages may be sent to the patient if the patient does not access the URL or complete the forms in a timely manner in block 316.

FIG. A illustrates a block diagram of an example method for receiving user data. In this regard, in block 402 the system 100 (of FIG. 1) receives an indication that the URL generated in block 312 (of FIG. 3) and sent to the user in block 314 has been accessed by the user. The system 100 authenticates the user identity by using verifiable personally identifiable data.

In block 404, the system 100 maintains a log in memory that includes whether the user has accessed the URL and maintains a record of the interactions the user has with the system 100. The log may be used to save the progress of a user. The user may pause their data entry process to begin at another time, or may ask the system 100 to prompt or remind the user to complete the tasks at another time, which may be specified by the user. The system may communicate with the user in a first language chosen by the user. The language is used during conversations with conversational AI (described below) as well as forms and documents presented to the user. The conversational AI may switch languages during a conversation with a user if desired by the user.

In block 406, the system 100 determines and selects interfaces for the fields in a form. For example the system 100 may use a mobile web form UI that includes, for example, radio buttons, pull-down menus, checkboxes. In embodiments, a conversational chat interface may be used. In block 408, when a conversational chat interface is selected, the system identifies the data types for data fields.

In block 410, the system 100 using the conversational chat interface engages in dialog with the user to collect information from the user. The conversational chat interface uses an artificial intelligence (AI) system that includes a large language model (LLM) to understand and to respond to user inputs. Users may interact with the system 100 using natural language. Based on the user input, the AI system may ask follow-up questions, gather more information, or conclude the dialogue. If the system 100 determines that the preferred data may not be collected the system may, for example, notify a human operator, or flag the matter for, for example a follow-up at a later time. The system 100 maintains context from the ongoing conversation and, in embodiments past conversations and electronic health records (EHRs) that may provide personalized dialogue tailored to the user and the history of the user. Context from a previous or ongoing conversation may include, for example, language, content from previous conversations, user-identifying data, user responses, and background information associated with the conversation.

In block 412 a question is presented to a user. The question may be presented on a screen or provided by audio. In block 414, the user inputs are saved in memory such as, for example, a stack arrangement.

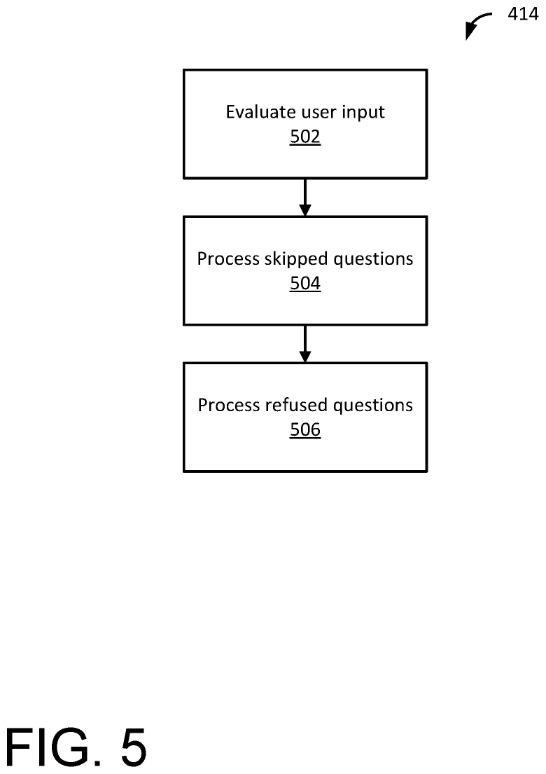

FIG. 5 illustrates a block diagram of the method described in block 414 (of FIG. A) In this regard, the system 100 using AI evaluates the inputs of the user to determine whether the input meets defined criteria. For example, if a birthdate of the user is requested, the user may be prompted to provide the data before presenting another question. For incomplete information, such as, for example, a missing year from a date, the system 100 retains the partial data (from the incomplete date), and prompts the user for the remainder of the data and pushes the next question and answer onto the stack. The system 100 may prompt the user multiple times for the correct information prior to progressing to another question. A persistence level may be set by a user that indicates how many times the system should prompt the user for data associated with a question before progressing to another question on the stack.

Questions skipped by a user are processed in block 504. In this regard, a user may skip a question if they do not readily possess the information for the answer. In such a circumstance, the questions may be presented by the system 100 to the user at a later time. In embodiments users may select the periodicity or next time the system should send a reminder to the user.

Refused questions by a user are processed in block 504. Users may refuse to answer questions for, for example, privacy reasons. If a user declines to answer a question for privacy reasons, the system 100 may provide context such as reasons the organization is collecting the information, which may convince the user to enter the data.

Referring to FIG. a unexpected patient responses and queries are processed in block 416 using LLMs and the system knowledge of the matter details. For example, if a user asks how long it will take them to travel to the location of the appointment (receiving office), the system will use the address of the user saved in memory and estimate a reasonable response. If, for example, the system cannot provide a response the system would provide contact information for the user to contact the receiving office to request information. The unexpected questions are flagged or identified by the system 100 for analysis by the LLM to determine potential gaps in context.

Image-based input may be received and processed in block 418. The user may be prompted to upload a requested image; the system 100 classifies uploaded images (e.g. an image of a health insurance card) and processes the images using an AI trained to extract structured information from an image. The data from the image is entered into a form and the system 100 prompts the user to confirm the data from the image is correct.

In block 420 the system prompts the user until the comprehensive data for each field is collected.

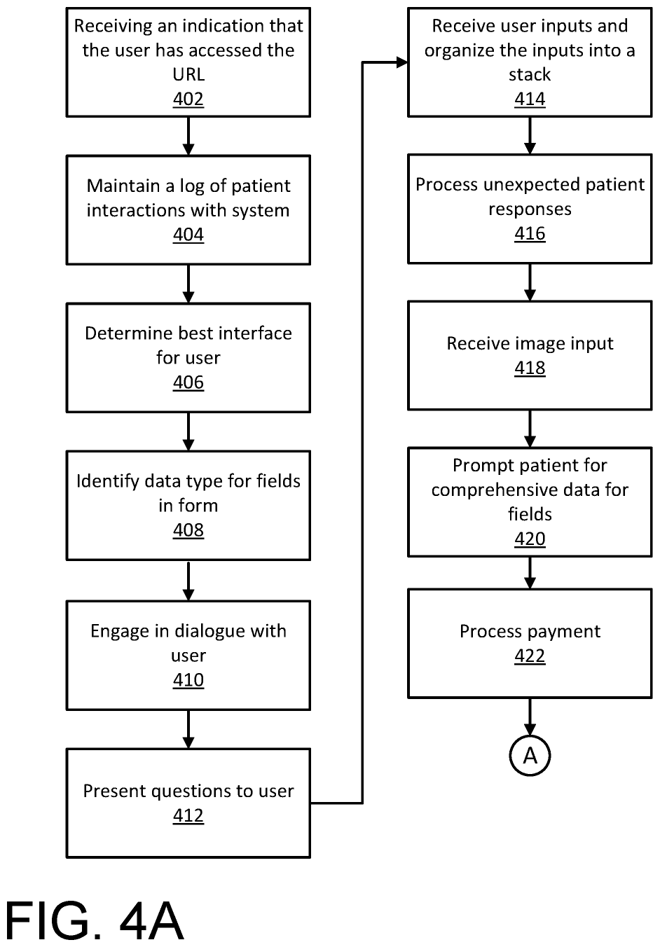

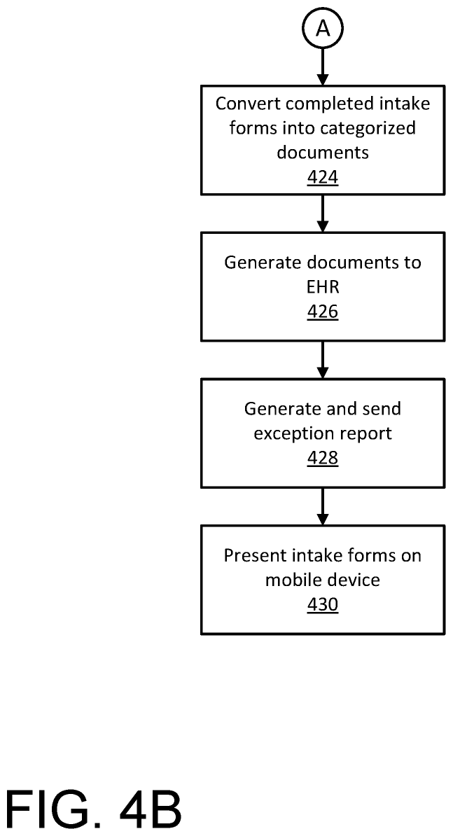

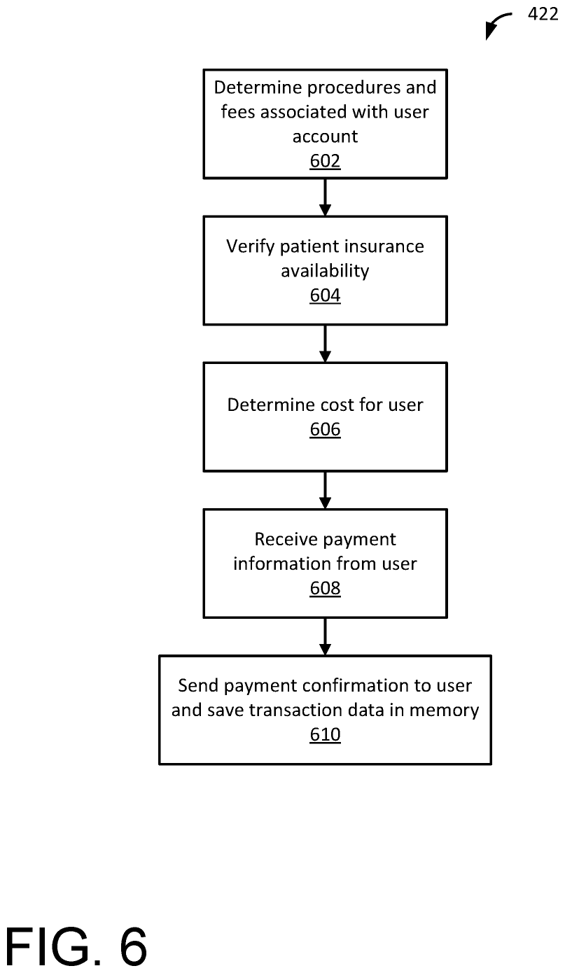

In block 422, the system 100 processes payments. FIG. 6 illustrates a block diagram of the method of processing payments described in block 422 (of FIG. 4). In this regard referring to FIG. 6, in block 602 billing procedures and fees associated with the user are determined. In block 604 the availability of insurance is verified. The out-of-pocket cost for the user is determined and presented to the user in block 606. In block 608, payment information from the user is received and processed. In block 610, the system 100 sends a payment confirmation to the user and saves the transaction in memory.

In block 424, once sufficient data has been collected, the system 100 converts the completed intake forms into documents (e.g. one or more documents) that are categorized using the file categories defined in the account of the user. File or document categories may include, for example, a health insurance card or health insurance information, identification, a family medical history, a list of medications, test results, and a consent form.

In block 426, the system 100 transmits the documents to an electronic health record (EHR) system via, for example, an API call, or HL7 standard according to the transmission rules associated with the account of the user.

When particular fields or forms remain incomplete, an exception report may be generated in block 428. In this regard, the exception report may be generated before an appointment date is set to allow the provider to contact the user to retrieve the data from the user. In block 430, a patient may complete additional intake forms or waivers in the office of the provider either via a link provided to the user on a mobile device.

The system described herein offers a method and system for collecting intake data from a user. The intake data may include any number of types of data. Previous forms provided on a computer or mobile device often fail to collect data from a patient in an efficient and effective manner.

The computer-readable program instructions may be received by a computer or processing device by any suitable process such as, for example, wireless, wired, or optical transmission via a network or other communications devices or processes.

Each block of the illustrations and block diagrams described herein can be implemented by computer-readable program instructions, and input to a processor of a computer to carry out or execute the computer-readable program instructions.

The descriptions of the various embodiments are disclosed for illustration but are not intended to limit the disclosed embodiments. Many modifications and variations will be apparent to those of ordinary skill in the art without departing from the scope of the described embodiments.

What is claimed is:

1. A method for receiving data, the method comprising:
responsive to receiving an indication that conversational artificial intelligence (AI) has been selected, identifying data types for a data field:
engaging in a dialog with a user using a large language model (LLM) to collect data for the data field wherein the dialog includes a question presented to the user and an answer provided by the user;
receiving data from the user;
tracking the user input on a stack;
providing a question to the user;
receiving an answer from the user;
determining if the answer is complete and meets the criteria for the data field; and
sending a second question to the user responsive to receiving a complete answer from the user.

2. The method of claim 1, wherein prior to receiving an indication that the conversational artificial intelligence has been selected, identifying data types for the data field:
sending a message to a user responsive to receiving an initiation trigger, wherein rules for initiating the initiation trigger are set by the user, the trigger rules include a message notifying the user that a period of time remains until an appointment of the user, or a request for the user to submit data.

3. The method of claim 1, wherein the method further includes:
converting the data and consent data into a document file;
sending the document file to an electronic health record (EHR) of the user;
attaching the document file to a chart of a patient.

4. The method of claim 1, wherein the dialog includes requesting data from a user using a conversational process, providing an additional request for data if the user did not provide sufficient or correct data for the data field.

5. The method of claim 1, wherein the AI evaluates the received data to determine whether the data meets a criteria associated with the data field;
if the data is incomplete, retaining the incomplete information and providing a reminder message to the user; and
sending a reminder message to the user if the user does not provide the requested data.

6. The method of claim 1, further comprising:
generating a context from content of the dialog from a past dialogue; and
providing input to the LLM that tunes the LLM to have a desired tone for the dialogue.

7. The method of claim 1, wherein the method further includes a method for a payment transaction from the user, the method includes:
identifying payment procedures in an account of the user;
verifying an insurance policy of the user;
determining the fees to be charged to the user;
displaying the fees to be charged to the user;
converting the data and consent data into a document file;
sending the document file to an electronic health record (EHR) of the user;
attaching the document file to a chart of a patient; and
logging the transaction and sending the transaction to the EHR of the user.

8. A system for receiving data, the system comprising:
a memory; and
a processor operative to:
responsive to receiving an indication that conversational artificial intelligence (AI) has been selected, identifying data types for a data field:
engage in a dialog with a user using a large language model (LLM) to collect data for the data field wherein the dialog includes a question presented to the user and an answer provided by the user;
receive data from the user;
track the user input on a stack;
provide a question to the user;
receive an answer from the user;
determine if the answer is complete and meets the criteria for the data field; and
send a second question to the user responsive to receiving a complete answer from the user.

9. The system of claim 8, wherein prior to receiving an indication that the conversational artificial intelligence has been selected, identifying data types for the data field:
sending a message to a user responsive to receiving an initiation trigger, wherein rules for initiating the initiation trigger are set by the user, the trigger rules include a message notifying the user that a period of time remains until an appointment of the user, or a request for the user to submit data.

10. The system of claim 8, wherein the method further includes:
converting the data and consent data into a document file;
sending the document file to an electronic health record (EHR) of the user;
attaching the document file to a chart of a patient

11. The system of claim 8, wherein the dialog includes requesting data from a user using a conversational process, providing an additional request for data if the user did not provide sufficient or correct data for the data field.

12. The system of claim 8, wherein the AI evaluates the received data to determine whether the data meets a criteria associated with the data field;
if the data is incomplete, retaining the incomplete information and providing a reminder message to the user; and
sending a reminder message to the user if the user does not provide the requested data.

13. The system of claim 8, further comprising:
generating a context from content of the dialog from a past dialogue; and
providing input to the LLM that tunes the LLM to have a desired tone for the dialogue.

14. The system of claim 8, wherein the method further includes a method for a payment transaction from the user, the method includes:
identifying payment procedures in an account of the user;
verifying an insurance policy of the user;
determining the fees to be charged to the user;
displaying the fees to be charged to the user;
converting the data and consent data into a document file;
sending the document file to an electronic health record (EHR) of the user;
attaching the document file to a chart of a patient; and
logging the transaction and sending the transaction to the EHR of the user.

15. A non-transitory computer-readable medium having a method stored therein, the method comprising:
responsive to receiving an indication that conversational artificial intelligence (AI) has been selected, identifying data types for a data field:
engaging in a dialog with a user using a large language model (LLM) to collect data for the data field wherein the dialog includes a question presented to the user and an answer provided by the user;
receiving data from the user;
tracking the user input on a stack;
providing a question to the user;
receiving an answer from the user;
determining if the answer is complete and meets the criteria for the data field; and
sending a second question to the user responsive to receiving a complete answer from the user.

16. The non-transitory computer-readable medium of claim 15, wherein the method further includes: prior to receiving an indication that the conversational artificial intelligence has been selected, identifying data types for the data field;
sending a message to a user responsive to receiving an initiation trigger, wherein rules for initiating the initiation trigger are set by the user, the trigger rules include a message notifying the user that a period of time remains until an appointment of the user, or a request for the user to submit data.

17. The non-transitory computer-readable medium of claim 15, wherein the method further includes:
converting the data and consent data into a portable document file;
sending the document file to an electronic health record (EHR) of the user;
attaching the document file to a chart of a patient.

18. The non-transitory computer-readable medium of claim 15, wherein the dialog includes requesting data from a user using a conversational process, providing an additional request for data if the user did not provide sufficient or correct data for the data field.

19. The non-transitory computer-readable medium of claim 15, the method further comprising:
generating a context from content of the dialog from a past dialogue; and
providing input to the LLM that tunes the LLM to have a desired tone for the dialogue.

20. The non-transitory computer-readable medium of claim 15, wherein the method further includes a method for a payment transaction from the user, the method includes:
identifying payment procedures in an account of the user;
verifying an insurance policy of the user;
determining the fees to be charged to the user;
displaying the fees to be charged to the user;
converting the data and consent data into a document file;
sending the document file to an electronic health record (EHR) of the user;
attaching the document file to a chart of a patient; and
logging the transaction and sending the transaction to the EHR of the user.
